(19) United States
(12) Patent Application Publication
(10) Pub. No.: US 2016/0331860 A1
(43) Pub. Date: Nov. 17, 2016

(54) WET WOUND DRESSING

(71) Applicant: TAICEND TECHNOLOGY CO., LTD, Kaohsiung City (TW)

(72) Inventors: Gou-Don CHU, Kaohsiung City (TW); Kun-Cheng TSAI, Kaohsiung City (TW)

(21) Appl. No.: 15/149,556

(22) Filed: May 9, 2016

(30) Foreign Application Priority Data
May 12, 2015 (TW) 104115023
Jan. 26, 2016 (TW) 105102408

(51) Int. Cl. A61L 15/26; A61L 15/42

(52) U.S. Cl. CPC A61L 15/26; A61L 15/425

(57) ABSTRACT

A wet wound dressing is formed from hydrophilic polyurethane foam. The hydrophilic polyurethane foam is formed by the foaming process using a hydrophilic polyurethane material and a foaming agent. The hydrophilic polyurethane material is star-shaped polyurethane formed by a reaction between a first polyether polyol and polyisocyanate then reacting with a second polyether polyol. The first polyether polyol includes at least three terminal hydroxyl groups. The hydrophilic polyurethane foam has non-continuous closed holes. The wet wound dressing possesses excellent permeability, water absorbency, and capable of preventing sticking of wound, thereby reducing frequency of changing wound dressing, such that wound healing is uninterrupted.

### CROSS-REFERENCE TO RELATED APPLICATION

This application claims priority from Taiwan Patent Application Nos. 104115023 and 105102408, filed respectively on May 12, 2015 and Jan. 26, 2016, in the Taiwan Intellectual Property Office, the contents of which are hereby incorporated by reference in its entirety for all purposes.

### BACKGROUND OF THE INVENTION

1. Field of the Invention

The present invention relates to a wound dressing; more specifically, the present invention relates to a wet wound dressing formed from the hydrophilic polyurethane foam.

2. Description of the Related Art

An ideal wound dressing should be capable of maintaining appropriate moisture around the wound while preventing scarring, cellular infiltration, and sticking of the wound to the dressing, without impeding new tissue growth. In particular, if the environment around the wound is maintained at a proper moisture level, the movement of the fibroblast can be sped up, thereby accelerating the formation of capillaries. In addition, the proper moisture level enables the wound to exude tissue fluid rich in collagen and growth factors capable of promoting cellular growth, such that wound healing may be accelerated. Besides, an ideal wound dressing does not require frequent changing, so the wound is able to stay in a closed, moist and yet permeable condition. Therefore the wound is free from infection caused by germs and capable of undergoing autolytic debridement.

However, methods known in the art such as natural healing, application of gauzes or cotton pads, etc. might cause the wound surface to dry up due to contact with air, thereby impeding the movement of fibroblast and exudation of tissue fluid. As a result, the healing of wounds is delayed and scarring might occur. On top of that, the wound healing might be further exacerbated since the changing of wound dressings e.g. gauzes might tear the healed tissues again. In order to maintain the moist environment around the wound and prevent the wound from sticking to the dressing, wound dressings such as artificial skin and foams are developed. However, due to the poor water absorption capability of the artificial skin, the wound might be soaked in excessive tissue fluid which causes cellular infiltration. On the other hand, the foam tends to stick to the wound due to the larger holes therein, despite having the ability to absorb water more effectively and maintain moisture level of the environment around the wound. Therefore, the dressings aforementioned apparently does not qualify as an ideal wound dressing and require frequent changing, thereby increasing the risk of wound infection due to germs and delaying the healing of wounds.

### SUMMARY OF THE INVENTION

In light of the aforementioned technical issues, an objective of the present invention is to provide a wet wound dressing capable of maintaining appropriate moisture around the wound while preventing scarring, cellular infiltration, and sticking of the wound to the dressing and does not require frequent changing.

According to an objective of the present invention, a wet wound dressing is provided. The wet wound dressing is formed from the hydrophilic polyurethane foam. The hydrophilic polyurethane foam is formed by the foaming process using a hydrophilic polyurethane material and a foaming agent. Wherein, the hydrophilic polyurethane material is star-shaped polyurethane formed by the reaction between a first polyether polyol and polyisocyanate then reacting with a second polyether polyol. The first polyether polyol includes at least three terminal hydroxyl groups. The hydrophilic polyurethane foam has non-continuous closed holes.

According to another objective of the present invention, a hydrophilic polyurethane material is provided. The hydrophilic polyurethane material is formed by the reaction between a first polyether polyol and polyisocyanate and then reacting with a second polyether polyol and hyaluronic acid to produce a star-shaped polyurethane block. The first polyether polyol includes at least three terminal hydroxyl groups.

Preferably, the polyisocyanate may be aliphatic polyisocyanate.

Preferably, the foaming agent may include blowing agents, water, surfactants, polyamines and catalysts.

Preferably, the average molar mass of hyaluronic acid may range from 500,000 to 2,500,000.

Preferably, the content of hyaluronic acid may make up 0.001-20 mol % of the sum of the first polyether polyol, polyisocyanate, second polyether polyol and hyaluronic acid. More preferably, the content of hyaluronic acid may make up 0.001-10 mol % of the aforementioned sum.

Preferably, the first polyether polyol may be polypropylene glycol triol (PPG triol).

Preferably, the second polyether polyol may be polyethylene glycol (PEG).

Preferably, the average molar mass of PEG may range from 1,000 to 6,000.

Preferably, the content of polyisocyanate may make up 20 to 70 mol % of the hydrophilic polyurethane material.

Preferably, the first polyether polyol and the second polyether polyol in the hydrophilic polyurethane foam may make up 20-70 mol % of the hydrophilic polyurethane material.

Preferably, the hydrophilic polyurethane foam may include a contact layer with a plurality of first holes and an absorption layer with a plurality of second holes. The contact layer and the absorption layer are integrally formed and the plurality of second holes are larger than the plurality of first holes.

Preferably, the wet wound dressing of the present invention may further include a permeation layer formed from the hydrophilic polyurethane material and disposed on a surface of the absorption layer. The content of the first polyether polyol and the second polyether polyol in the permeation layer may range from 20 to 70 mol % of the hydrophilic polyurethane material.

Preferably, the contact layer may be formed by scrapping the surface of the hydrophilic polyurethane foam and applying a predetermined pressure to the surface during the formation of the hydrophilic polyurethane foam.

Preferably, the predetermined pressure may range from 100 to 300 g/cm².

In conclusion, the wet wound dressing of the present invention possesses one or more advantages listed below:

1. Permeability: since the hydrophilic polyurethane material in the wet wound dressing of the present invention contains both hydrophilic and hydrophobic ends, there will be no cross-links between molecules due to the intermolecular repulsion force. Hence, there will be tiny gaps between molecules which permit the diffusion of air.

2. Water absorbency: since the star-shaped polyurethane in the wet wound dressing of the present invention has star-shaped structures with larger specific surface area, the water absorbency of the present wet wound dressing is promoted. Therefore, excessive tissue fluid can be absorbed to prevent cellular infiltration.

3. Prevention of wound sticking: the non-continuous closed holes in the wet wound dressing of the present invention prevent the cells from growing into the contact layer. Furthermore, the polyethylene glycol in the present invention is able to prevent protein and cell adhesion, thereby further preventing the wound from sticking to the wound dressing.

### BRIEF DESCRIPTION OF THE DRAWINGS

FIG. 1 is a schematic diagram illustrating the first embodiment of the wet wound dressing according to the present invention.

FIG. 2 is a schematic diagram illustrating the handling of the first embodiment of the wet wound dressing according to the present invention.

FIG. 3 is a sectional view illustrating the application of the second embodiment of the wet wound dressing on the wound according to the present invention.

FIGS. 4 to 6 are the sectional views illustrating different stages for the formation of the second embodiment of the wet wound dressing shown in FIG. 3 according to the present invention.

### DESCRIPTION OF THE PREFERRED EMBODIMENTS

Various aspects like the technical features, advantages or content of the present invention will be set forth in detail in the form of preferred embodiments hereinafter. Description will be made along with reference to the attached drawings, which are solely illustrative and serve to provide better understanding of the present invention only, and the scale and/or proportion of any portion of the drawing do not represent the actual configuration of the invention. Hence, the scale, proportion or shape in the drawings should not be misconstrued as limiting the scope of the invention

Hereinafter, the wet wound dressing of the present invention will be set forth with respect to the accompanying drawings; similar elements in the embodiments will be assigned with similar numerals for the clarity of the description.

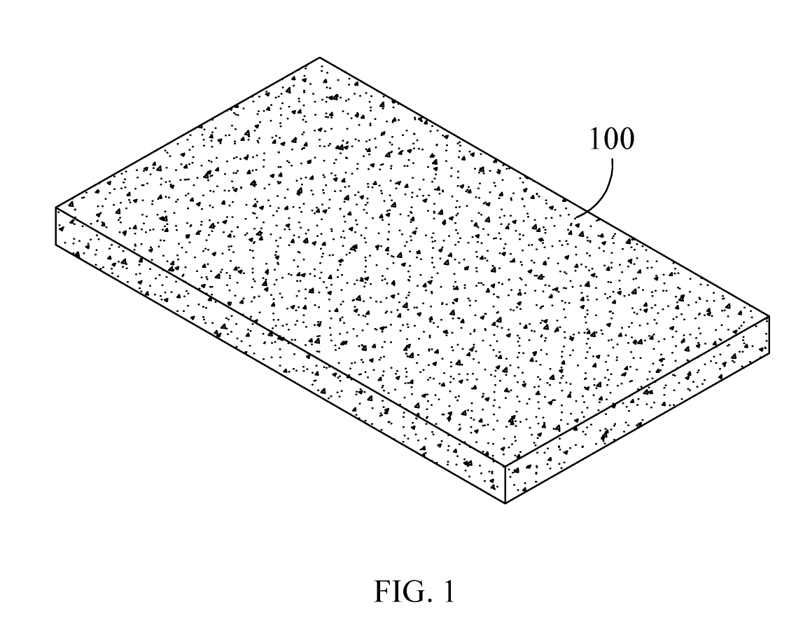

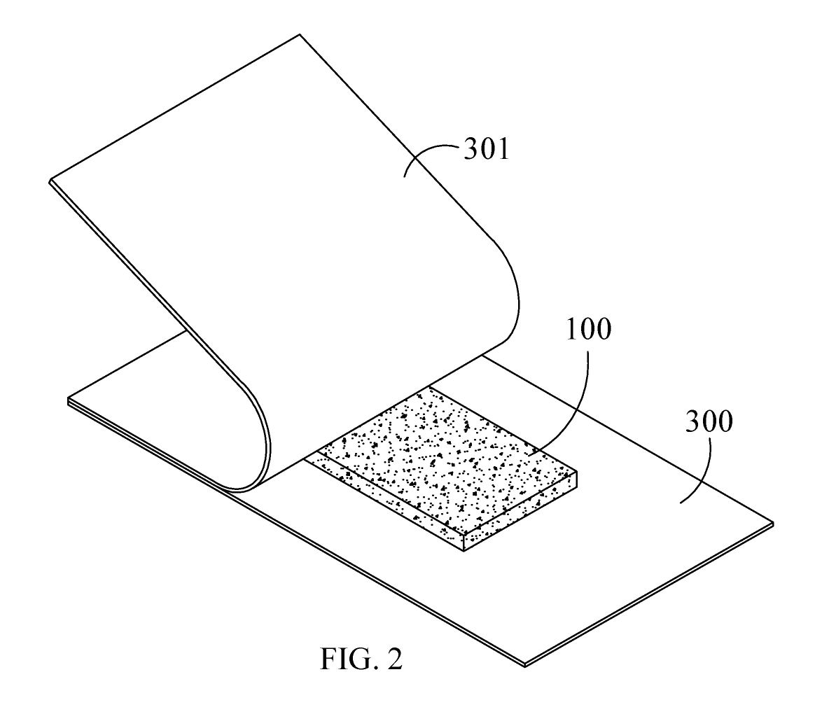

FIGS. 1 and 2 are schematic diagrams respectively illustrating the first embodiment of the wet wound dressing and the handling thereof according to the present invention. In the first embodiment of the present invention, the wet wound dressing may be formed from the hydrophilic polyurethane foam 100, but not limited thereto; the wet wound dressing may be formed directly from the hydrophilic polyurethane material without undergoing the foaming process. For the formation of the hydrophilic polyurethane material, the first polyether polyol with at least three terminal hydroxyl groups, preferably polypropylene glycol triol (PPG triol), is initially reacted with polyisocyanate, such that a star-shaped prepolymer is formed; subsequently, the star-shaped prepolymer is reacted with the second polyether polyol, preferably polyethylene glycol (PEG) and hyaluronic acid, such that the cross-linking reaction produces the star-shaped polyurethane block.

Preferably, polyisocyanate may be aliphatic polyisocyanate, since aromatic polyisocyanate may be toxic. More preferably, the aliphatic polyisocyanate may be selected from the group consisting of hexamethylene diisocyanate (HDI), methylene dicyclohexyl diisocyanate (H₁₂MDI) and isophorone diisocyanate (IPDI) or the mixture thereof. Moreover, the preferable average molar mass PEG may range from 1,000 to 6,000. Wherein, metabolite of PEG with molar mass below 1,000 might exhibit biotoxicity. On the other hand, if the molar mass of PEG exceeds 6,000 the cross-linking reaction might be unmanageable due to high viscosity thereof. If solvent is added to PEG to better manage the cross-linking reaction, there might be residual solvent in the dressing which possesses cytotoxicity. In addition, the ideal average molar mass of hyaluronic acid may range from 500,000 to 2,500,000, since hyaluronic acid with average molar mass above 2,500,000 is not conducive for wound healing.

Next, the hydrophilic polyurethane material is reacted with a foaming agent such that the hydrophilic polyurethane foam 100 is formed from the foaming process. The foaming agent may include blowing agents, water, surfactants, polyamines and catalysts. Wherein the polyamine enhances the mechanical strength of the hydrophilic polyurethane material. Preferably, polyamine may be selected from the group consisting of ethane-1,2-diamine, butane-1,4-diamine, hexane-1,6-diamine, triethylenetetramine (TETA) and polyetheramine Wherein polyetheramine may be selected from the JEFFAMINE® manufactured by Huntsman Corporation, but not limited thereto. The catalyst serves to catalyze the reaction between excess of polyisocyanate and water to produce carbon dioxide. Preferably, the catalyst may be selected from the group consisting of zinc octoate, triethylenediamine (TEDA, DABCO), dimethylcyclohexylamine (DMCHA), dimethylethanolamine (DMEA), trimethylamine (TEA), 1,8-Diazabicyclo[5.4.0]undec-7-ene (DBU) and pentamethyldiethylenetriamine (PMDETA).

It is noteworthy that the formation of the holes of the hydrophilic polyurethane foam 100 is achieved by adding excess of polyisocyanate and water such that the reaction produces carbon dioxide (CO₂); and then adding the foaming agent such that the foaming reaction causes the hydrophilic polyurethane material to undergo self-foaming, and finally the holes are formed. Wherein, the content of the polyisocyanate may range from 20-70 mol % of the hydrophilic polyurethane material; preferably 40-50 mol %. As a result non-continuous closed holes can be formed inside the hydrophilic polyurethane foam 100, wherein the holes are mutually independent of each other.

Hereinafter, detailed descriptions of the wet wound dressing of the present invention will be given with reference to the exemplary products and the exemplary embodiments, and the wound healing test conducted using the exemplary embodiments and the comparative embodiments.

Exemplary Product 1: Star-shaped Hydrophilic Polyurethane PU1

1 mol of PPG6000 triol (average molar mass is 6,000) is mixed with 3 mol of HDI, the reaction is kept at 80° C. for 1 hour, and then the star-shaped prepolymer is obtained; after that 1 mol of PEG1000 (average molar mass is 1,000), 1.5 mol of PEG2000 (average molar mass is 2,000) and 0.5 mol of hyaluronic acid (average molar mass is 1,000,000) are added to the star-shaped prepolymer, and the cross-linking process is kept at 80° C. for 6 hours, finally the star-shaped hydrophilic polyurethane PU1 is obtained.

Exemplary Product 2: Star-shaped Hydrophilic Polyurethane PU2

0.5 mol of PPG6000 triol is mixed with 2.5 mol of HDI, the reaction is kept at 80° C. for 1 hour, and then the star-shaped prepolymer is obtained; after that 0.85 mol of PEG1000, 0.85 mol of PEG2000 and 0.1 mol of hyaluronic acid (average molar mass is 1,000,000) are added to the star-shaped prepolymer, and the cross-linking process is kept at 80° C. for 6 hours, finally the star-shaped hydrophilic polyurethane PU2 is obtained.

### Exemplary Embodiment 1

### Wet Wound Dressing DR1

The star-shaped hydrophilic polyurethane PU1 of the exemplary product 1 is mixed with 1 mol of HDI, the mixture is stirred and then applied on a release film, apply heating of 120° C. for 1 hour, and the wet wound dressing DR1 of the present invention is obtained.

### Exemplary Embodiment 2

### Wet Wound Dressing DR2

0.1 mol of sodium bicarbonate (blowing agent), 0.4 mol of water, 0.5 mol of polydimethylsiloxane-polyoxyethylene copolymer (surfactant), 0.1 mol of ethylenediamine, and 0.1 mol of zinc octoate (catalyst, purchased from TMG Chemicals Taiwan, model no. TMG620) are mixed to form a foaming agent, and then the star-shaped hydrophilic polyurethane PU2 of the exemplary product 2 is quickly mixed and stirred with the foaming agent; after the completion of foaming, the hydrophilic polyurethane foam 100 is obtained. The holes in the hydrophilic polyurethane foam 100 are closed holes that are non-continuous. Subsequently, as illustrated by FIG. 2, the hydrophilic polyurethane foam 100 is cut into appropriate sizes and adhered to the adhesive backing pad 300, then the hydrophilic polyurethane foam 100 and the backing pad 300 are covered with the release paper 301, and finally the wet wound dressing DR2 is obtained.

### Comparative Embodiment 1

### Wound Dressing CDR1

The wound dressing CDR1 of the comparative embodiment 1 is a common gauze.

### Comparative Embodiment 2

### Wound Dressing CDR2

The wound dressing CDR2 of the comparative embodiment 2 is the Soft Cloth Dressing with Pad 3662A marketed by 3M Company, wherein the material thereof contacting the wound is non-woven fabric.

### Comparative Embodiment 3

### Wet Wound Dressing CDR3

The wet wound dressing CDR3 of the comparative embodiment 3 is the hydrogel wound dressing HERADERM® marketed by AMED Co. Ltd. Taiwan. The material thereof contacting the wound is 2-hydroxyethyl methacrylate (HEMA).

Wound Healing Test

A. Animals Used for Experiment:

The experiment hereinafter used male Sprague-Dawley (S.D.) rats that are 8 weeks old, and weigh approximately 200 g. All experimented animals are kept in the animal experiment facility with photoperiod of 12 hours each for day and night, room temperature of 22° C. and relative humidity at 42%; each experimented animal is provided with adequate amount of water and animal feed. Before the conduction of experiment, the animals are given at least 2 weeks of time to adapt to the environment. All aspects of welfare pertaining to the keeping condition and handling of the experimented animals and the experimental procedures on the animals in its entirety are in accordance with the Guide for the Care and Use of Laboratory Animals set by the National Institutes of Health (NIH).

B. Sterilization of the Wound Dressings:

The wound dressings DR1, DR2 and CDR1 obtained from the exemplary embodiments 1 and 2 and comparative embodiment 1 are subjected to gamma ray of 40k Gy for sterilization, and then the experiment as follows is carried out.

C. Skin Wound Formation:

The dorsal part of the S.D. rats is shaved, and then disinfected with tincture of iodine and 70% alcohol. Subsequently, a skin wound of approximately 2 cm×2 cm in area and approximately 2 to 3 mm in depth is carved on the left dorsal of the S.D. rats with the surgical knife.

D. Application of Dressings:

The S.D. rats are randomly distributed into 2 experimental groups and 3 control groups, i.e. Experimental Groups 1 and 2 and Control Groups 1, 2 and 3, where each group contains 3 S.D. rats. Every S.D. rat in each group has a skin wound formed using the skin wound formation method described in the foregoing section C. Then the wet wound dressings DR1 and DR2 sterilized according to the method described in the foregoing section B are respectively applied to the skin wound of the S.D. rats in the Experimental Groups 1 and 2. On the other hand, the wound dressing CDR1 sterilized according to the method described in the foregoing section B, wound dressing CDR2 and the wet wound dressing CDR3 are respectively applied to the skin wound of the S.D. rats in the Control Groups 1, 2 and 3. The experiment lasts for 7 days, the skin wound area of the S.D. rats in each group is measured on 1^{st}, 3^{rd}, 5^{th}, and 7^{th} day after the application of wound dressings, and the results are tabulated in Table 1.

TABLE 1

| | 1^{st} day | | 3^{rd} day | | 5^{th} day | | 7^{th} day | |
|---|---|---|---|---|---|---|---|---|
| | after | | after | | after | | after | |
| | applying | | applying | | applying | | applying | |
| | wound | | wound | | wound | | wound | |
| | dressing | | dressing | | dressing | | dressing | |
| Experimental | 3.53 | cm² | 2.22 | cm² | 1.10 | cm² | 0.12 | cm² |
| Group 1 | | | | | | | | |
| Experimental | 3.24 | cm² | 1.21 | cm² | 0.16 | cm² | 0.00 | cm² |
| Group 2 | | | | | | | | |
| Control Group 1 | 4.00 | cm² | 3.61 | cm² | 2.89 | cm² | 2.25 | cm² |
| Control Group 2 | 4.00 | cm² | 2.89 | cm² | 1.96 | cm² | 1.00 | cm² |
| Control Group 3 | 3.61 | cm² | 2.25 | cm² | 1.21 | cm² | 0.36 | cm² |

As can be appreciated in table 1, the skin wound area of the S.D. rats in Experimental Groups 1 and 2 has shrunk significantly after applying dressings for 1 to 7 days. Particularly, the skin wound of the S.D. rats in Experimental Group 2 is almost healed on the 7^{th} day after applying wound dressing. On the contrary, the skin wound of the S.D. rats in the Control Groups 1 to 3 heals in a significantly slower pace. The results in Table 1 evidently shows that the wet wound dressings DR1 and DR2 of the Experimental Groups 1 and 2 have far superior wound healing effect in contrast to the wound dressings CDR1-CDR3 of the Control Groups 1-3.

The first embodiment of the wet wound dressing of the present invention has better water absorbency attributed to the star-shaped polyurethane block thereof having larger specific surface area, thereby capable of absorbing excessive tissue fluid and preventing cellular infiltration. Besides, with the help of non-continuous closed holes and PEG capable of preventing protein and cell adhesion, the cell will not grow into the wound dressing, thus sticking of wound can be prevented. Besides, the wet wound dressing of the present invention does not contain monomers or cross-linking agents that may be toxic, therefore the biosafety is promoted.

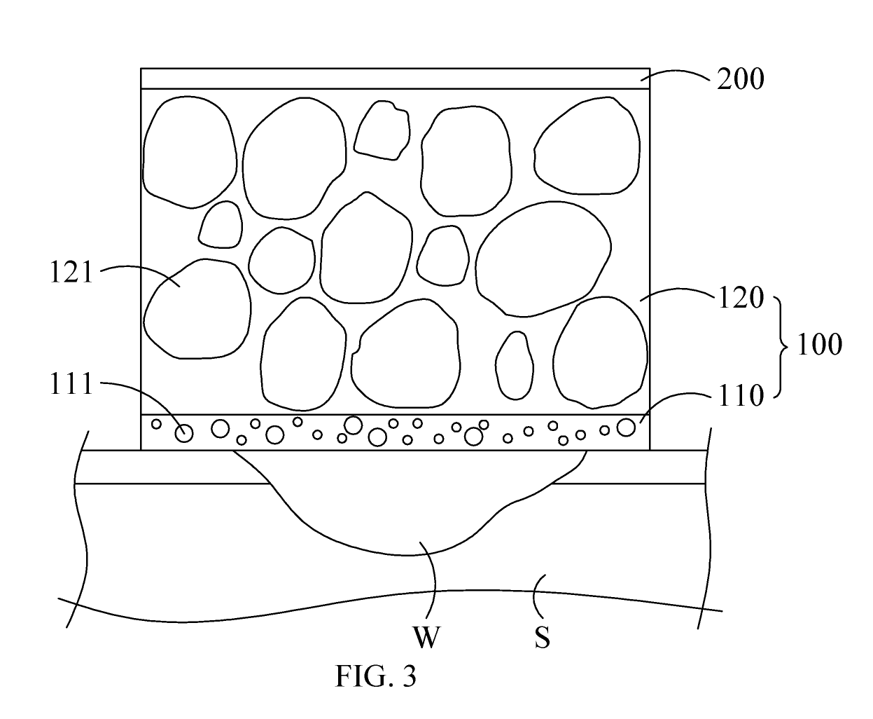

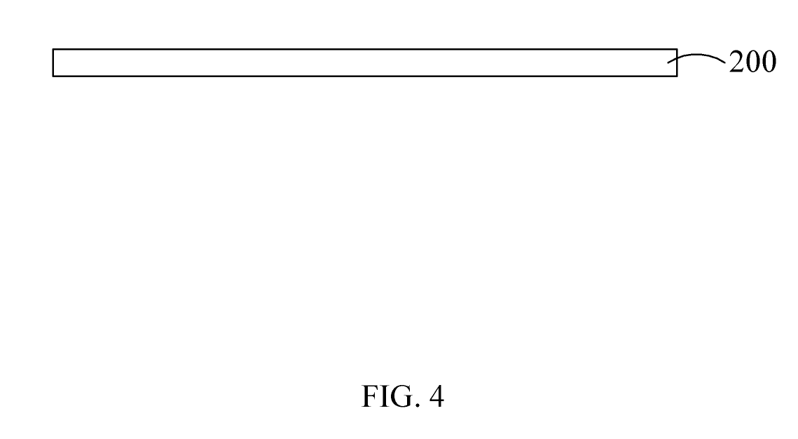

Referring to FIGS. 3 to 6, in which FIG. 3 is a sectional view illustrating the application of the second embodiment of the wet wound dressing on the wound, while FIGS. 4 to 6 are the sectional views illustrating different stages for the formation of the second embodiment of the wet wound dressing shown in FIG. 3 according to the present invention. In the second embodiment of the present invention, the wet wound dressing may include the hydrophilic polyurethane foam 100 and the permeation layer 200. Wherein, the hydrophilic polyurethane foam 100 may further include the contact layer 110 and the absorption layer 120 that are formed integrally. As shown in the FIG. 3, the contact layer 110 is disposed with a plurality of first holes 111, and one surface of the contact layer 110 is in contact with the wound W. The absorption layer 120 having a plurality of second holes 121 is disposed on the other surface of the contact layer 110, wherein the plurality of second holes 121 are larger than the plurality of first holes 111. The permeation layer 200 formed from the hydrophilic polyurethane material is disposed over the absorption layer 120.

More specifically, as shown in FIG. 4, 1 mol of first polyether polyol is mixed with 3 mol of polyisocyanate, and then the mixture is allowed to react at 70° C. for 2 hours to produce a prepolymer. Subsequently, 3 mol of second polyether polyol is added to the prepolymer and the mixture is allowed to react at 40° C. for 0.1 hour to yield the hydrophilic polyurethane material. The aforementioned hydrophilic polyurethane material is made into the permeation layer 200, for instance but not limited to polyurethane film. The content of the first polyether polyol and the second polyether polyol in the permeation layer 200 may range from 20 to 70 mol % of the hydrophilic polyurethane material, preferably range from 40-50 mol %; such that the permeation layer 200 with both hydrophilic ends and hydrophobic ends is formed. Since the hydrophilic end and the hydrophobic end are mutually repulsive, tiny pores only permeable to air can be formed, thereby endowing the present invention with excellent permeability. Besides, the size of the tiny pores may be smaller than that of the water molecule, such that the water molecules are incapable of entering the permeation layer 200. Therefore permeation layer 200 is waterproof and capable of inhibiting the growth of microbes, thereby preventing exogenous infection.

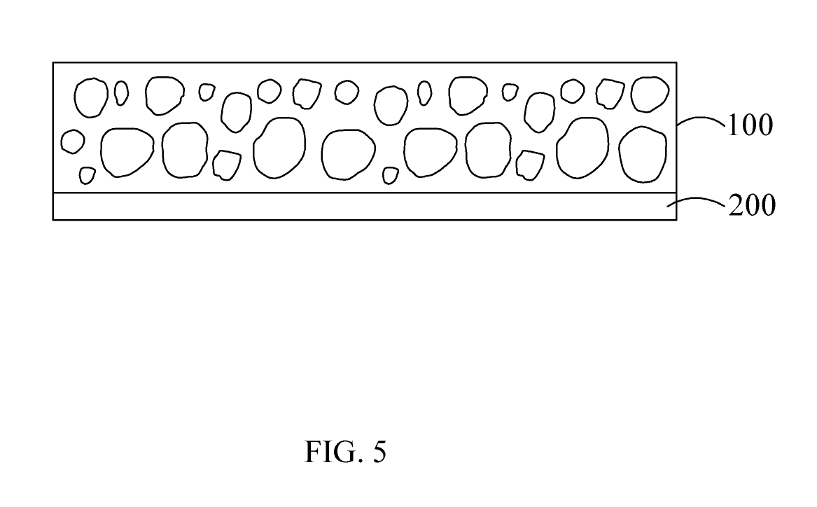

Then, as illustrated by the FIG. 5, 1 mol of first polyether polyol is mixed with 3 mol of polyisocyanate, and then the mixture is allowed to react at 70° C. for 2 hours to produce a star-shaped prepolymer. Subsequently, 3 mol of second polyether polyol is added to the prepolymer and the mixture is allowed to react at 40° C. for 0.1 hour to produce the star-shaped hydrophilic polyurethane. The aforementioned 4 mol of star-shaped hydrophilic polyurethane is briskly stirred with the foaming agent and the mixture is applied on the permeation layer 200, thereby integrally forming the hydrophilic polyurethane foam 100 and the permeation layer 200. Particularly, the holes in the hydrophilic polyurethane foam 100 are closed and non-continuous and the content of the first polyether polyol and the second polyether polyol therein may range from 20 to 70 mol % of the hydrophilic polyurethane material, preferably 40-50 mol %.

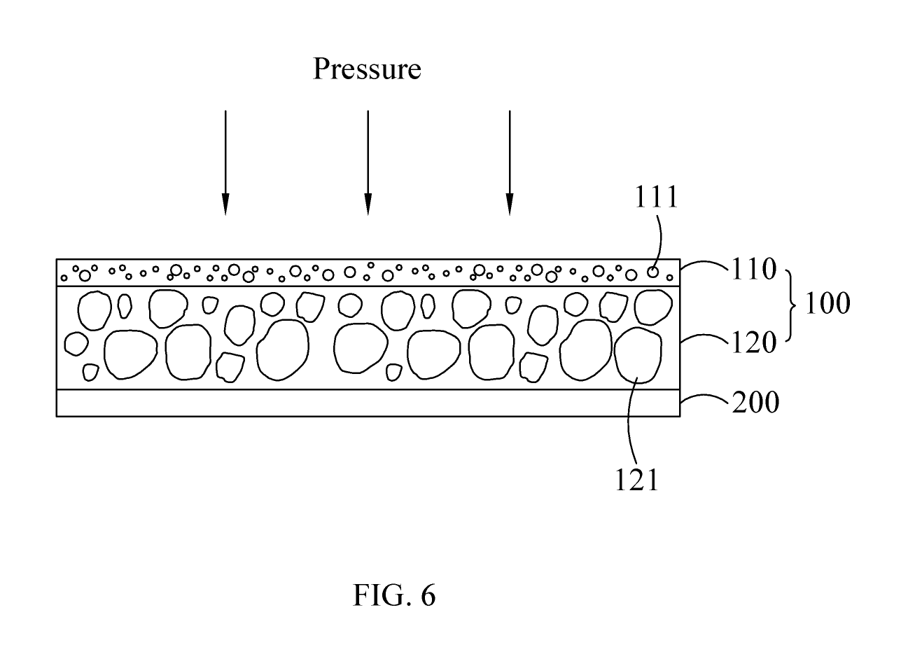

Next, as shown in the FIG. 6, during the formation of the hydrophilic polyurethane foam 100 i.e. before the completion of the foaming process, the surface of the hydrophilic polyurethane foam 100 may be subjected to actions such as scrapping, but not limited thereto; and subjected to pressure of 100 to 300 g/cm², preferably 120-250 g/cm², in order to compress the bubbles formed at the surface of the hydrophilic polyurethane foam 100 during the foaming process. Therefore the contact layer 110 having the first holes 111 with smaller diameter is formed on the surface of the hydrophilic polyurethane foam 100. The diameter of the first holes 111 is preferably smaller than that of the cells, e.g. 40-140 μm, preferably, 40-60 μm. The absorption layer 120 is interposed between the contact layer 110 and the permeation layer 200, and disposed with the second holes 121 larger than the first holes 111. The diameter of the second holes 121 may range from 100 to 1000 μm, preferably 400-800 μm. Wherein the thickness of the contact layer 110 may make up 0.1-5%, preferably 0.1-1% of the overall thickness of the hydrophilic polyurethane foam 100.

It is noteworthy that the first holes 111 i.e. non-continuous closed holes smaller than cell diameter and the PEG capable of preventing protein and cell adhesion help prevent the cells in the wound W from growing into contact layer 110, thereby preventing the wound from sticking to the wound dressing. Besides, since the second holes 121 of the absorption layer 120 are able to absorb large quantity of tissue fluid, the moisture around the wound W is maintained at a proper level without causing cellular infiltration. As a result, the movement of the fibroblast can be accelerated to promote capillaries formation as well as accelerate the decomposition of necrotic tissue and fibrin. In addition, after absorbing tissue fluid, the second holes 121 will expand and deform, so the present invention is able to adhere firmly to the wound W and skin S, and the contraction of fibroblast towards the center of the wound W during the healing can be avoided to prevent scarring.

In conclusion, the wet wound dressing of the present invention possesses excellent permeability owing to the material therein having both hydrophilic and hydrophobic ends; besides the star-shaped polyurethane has exceptional water absorbency, so the wet wound dressing is capable of preventing cellular infiltration and maintaining the moisture level around the wound at the optimum level. Moreover, the contact layer of the wet wound dressing contains PEG and non-continuous closed holes; wherein PEG can prevent protein and cell adhesion; and the diameter of the non-continuous closed holes is smaller than that of cell, such that the wound will not stick to the wet wound dressing of the present invention. Therefore, the wet wound dressing of the present invention can be used for 7 to 14 days without changing, so autolytic debridement is not interrupted and the wound healing can be accelerated.

The descriptions hereinbefore are merely illustrative instead of restrictive. It is understood that various modifications could be applied to the disclosure without deviating from the scope and spirit of the invention that is set forth in the appended claims.

What is claimed is:

1. A wet wound dressing formed from a hydrophilic polyurethane foam; the hydrophilic polyurethane foam being formed by a foaming process using a hydrophilic polyurethane material and a foaming agent;
wherein the hydrophilic polyurethane material is star-shaped polyurethane formed by a reaction between a first polyether polyol and polyisocyanate then reacting with a second polyether polyol; the first polyether polyol comprises at least three terminal hydroxyl groups; and the hydrophilic polyurethane foam has non-continuous closed holes.

2. The wet wound dressing of claim 1, wherein the hydrophilic polyurethane foam comprises a contact layer with a plurality of first holes and an absorption layer with a plurality of second holes; the contact layer and the absorption layer are integrally formed and the plurality of second holes are larger than the plurality of first holes.

3. The wet wound dressing of claim 1, wherein the foaming agent comprises blowing agents, water, surfactants, polyamines and catalysts.

4. The wet wound dressing of claim 1, wherein the first polyether polyol is polypropylene glycol triol.

5. The wet wound dressing of claim 1, wherein the second polyether polyol is polyethylene glycol.

6. The wet wound dressing of claim 1, wherein a content of polyisocyanate ranges from 20 to 70 mol % of the hydrophilic polyurethane material.

7. The wet wound dressing of claim 2, wherein a content of the first polyether polyol and the second polyether polyol in the hydrophilic polyurethane foam ranges from 20 to 70 mol % of the hydrophilic polyurethane material.

8. The wet wound dressing of claim 2, further comprising a permeation layer formed from the hydrophilic polyurethane material and disposed on a surface of the absorption layer, wherein a content of the first polyether polyol and the second polyether polyol in the permeation layer ranges from 20 to 70 mol % of the hydrophilic polyurethane material.

9. The wet wound dressing of claim 2, wherein the contact layer is formed by scrapping a surface of the hydrophilic polyurethane foam and applying a predetermined pressure to the surface during the formation of the hydrophilic polyurethane foam.

10. The wet wound dressing of claim 9, wherein the predetermined pressure ranges from 100 to 300 g/cm².
